(19) United States
(12) Patent Application Publication
(10) Pub. No.: US 2007/0178489 A1
(43) Pub. Date: Aug. 2, 2007

(54) Compositions and Methods for Enhanced Sensitivity and Specificity of Nucleic Acid Synthesis

(75) Inventors: Mekbib Astatke, Gaithersburg, MD (US); Deb K. Chatterjee, North Potomac, MD (US); Harini Shadilya, Bethesda, MD (US)

Correspondence Address: INVITROGEN CORPORATION;C/O INTELLEVATE, P.O. BOX 52050, MINNEAPOLIS, MN, 55402, US

(73) Assignee: Invitrogen Corporation, Carlsbad, CA (US)

(21) Appl. No.: 11/539,347

(22) Filed: Oct. 6, 2006

Related U.S. Application Data
(63) Continuation of application No. 09/570,526, filed on May 12, 2000, now abandoned.
(60) Provisional application No. 60/133,860, filed on May 12, 1999.

(51) Int. Cl. C12Q 1/68; C12P 19/34

(52) U.S. Cl. 435/6; 435/91.2

(57) ABSTRACT

The present invention relates to polypeptides, compositions and methods for enhancing synthesis of nucleic acid molecules. In a preferred aspect, the invention relates to inhibition or control of nucleic acid synthesis, sequencing or amplification. Specifically, the present invention discloses polypeptides having affinity for double-stranded and/or single-stranded nucleic acid molecules and/or single-stranded/double-stranded nucleic acid complexes (e.g., primer/template complexes, double-stranded templates, single-stranded templates or single-stranded primers) for use in such enhanced synthesis and more particularly to polymerases having reduced polymerase and optionally reduced exonuclease activities (3′ to 5′ and/or 5′ to 3′ exonuclease activity), and to nucleases having reduced nuclease activity. The polypeptides of the invention are capable of inhibiting nonspecific nucleic acid synthesis at ambient temperature. Thus, in a preferred aspect, the invention relates to “hot start” synthesis of nucleic acid molecules. Accordingly, the invention prevents non-specific nucleic acid synthesis at low temperatures, for example during reaction set up. The invention also relates to kits for synthesizing, amplifying, reverse transcribing or sequencing nucleic acid molecules comprising one or more of the polypeptides or compositions of the invention. The invention also relates to compositions prepared for carrying out the methods of the invention and to compositions made after or during such methods. The invention also generally relates to polypeptides and compositions useful for inhibiting or preventing degradation of various nucleic acid molecules.

### RELATED APPLICATIONS

This application is a continuation-in-part of U.S. application Ser. No. 60/13,860 filed May 12, 1999, the entire contents of which are incorporated herein by reference.

### FIELD OF THE INVENTION

The present invention relates to a method for increasing sensitivity and specificity of nucleic acid synthesis by reducing nonspecific nucleic acid synthesis which may occur for example at ambient temperatures. The invention also relates to compositions and polypeptides for carrying out the methods of the invention. The methods and compositions of the present invention can be used in sequencing, amplification reactions, nucleic acid synthesis and cDNA synthesis.

The invention also relates to polypeptides and compositions which are capable of inhibiting or preventing nucleic acid synthesis, sequencing, amplification and cDNA synthesis, for example, by binding one or more double-stranded nucleic acid molecules and/or single stranded nucleic acid molecules and/or double-stranded single-stranded complexes. Thus the invention may inhibit or prevent nucleic acid synthesis, sequencing, amplification, and cDNA synthesis reactions by binding or interacting with nucleic acid substrates used in such reactions (e.g., primers, templates and primer/template complexes). The invention also relates to polypeptides and compositions which are capable of inhibiting or preventing degradation of nucleic acid molecules (preferably single-stranded molecules or single-stranded containing molecules) by binding or interacting with such molecules. Such interaction preferably prevents or inhibits degradation of the nucleic acid molecules with nucleases, particularly exonucleases and specifically single-stranded specific exonucleases. The invention also relates to nucleic acid molecules encoding the polypeptides of the invention, and to vectors and host cells comprising such nucleic acid molecules.

The invention also concerns kits comprising the compositions or polypeptides of the invention.

### BACKGROUND OF THE INVENTION

DNA polymerases synthesize the formation of DNA molecules which are complementary to all or a part of a DNA template. Upon hybridization of a primer to the single-stranded DNA template, polymerases synthesize DNA in the 5′ to 3′ direction, successively adding nucleotides to the 3′-hydroxyl group of the growing strand. Thus, in the presence of deoxyribonucleoside triphosphates (dNTPs) or nucleotides and a primer, a new DNA molecule, complementary to all or a part of the single stranded DNA template, can be synthesized.

Both mesophilic and thermophilic DNA polymerases are used to synthesize the formation of nucleic acids. In PCR or cycle sequencing, using thermostable rather than mesophilic polymerase is preferable due to the reduced level of non-specific DNA amplification that results from extending mis-annealed primer termini at less stringent annealing temperatures, e.g. ambient temperature. However, for some primer sequences and under certain experimental conditions significant amounts of synthesis of non-specific nucleic acid products reduce the sensitivity of the thermostable polymerase, requiring extensive optimization for each primer set. In addition, this problem is intensified when polymerases having high level activity at ambient temperature are employed (for example, DNA polymerase from Thermatoga neapolitana).

In examining the structure and physiology of an organism, tissue or cell, it is often desirable to determine its genetic content. The genetic framework of an organism is encoded in the double-stranded sequence of nucleotide bases in the deoxyribonucleic acid (DNA) which is contained in the somatic and germ cells of the organism. The genetic content of a particular segment of DNA, or gene, is only manifested upon production of the protein which the gene encodes. In order to produce a protein, a complementary copy of one strand of the DNA double helix (the “coding” strand) is produced by polymerase enzymes, resulting in a specific sequence of ribonucleic acid (RNA). This particular type of RNA, since it contains the genetic message from the DNA for production of a protein, is called messenger RNA (mRNA).

Within a given cell, tissue or organism, there exist many mRNA species, each encoding a separate and specific protein. This fact provides a powerful tool to investigators interested in studying genetic expression in a tissue or cell. mRNA molecules may be isolated and further manipulated by various molecular biological techniques, thereby allowing the elucidation of the full functional genetic content of a cell, tissue or organism.

A common approach to the study of gene expression is the production of complementary DNA (cDNA) clones. In this technique, the mRNA molecules from an organism are isolated from an extract of the cells or tissues of the organism. This isolation often employs chromatography matrices, such as cellulose or agarose, to which oligomers of thymidine (T) have been complexed. Since the 3′ termini on most eukaryotic mRNA molecules contain a string of adenosine (A) bases, and since A binds to T, the mRNA molecules can be rapidly purified from other molecules and substances in the tissue or cell extract. From these purified m-RNA molecules, cDNA copies may be made using the enzyme reverse transcriptase (RT) or DNA polymerases having RT activity, which results in the production of single-stranded cDNA molecules. The single-stranded cDNAs may then be converted into a complete double-stranded DNA copy (i.e., a double-stranded cDNA) of the original mRNA (and thus of the original double-stranded DNA sequence, encoding this mRNA, contained in the genome of the organism) by the action of a DNA polymerase. The protein-specific double-stranded cDNAs can then be inserted into a vector, which is then introduced into a host bacterial, yeast, animal or plant cell, a process referred to as transformation or transfection. The host cells are then grown in culture media, resulting in a population of host cells containing (or in many cases, expressing) the gene of interest or portions of the gene of interest.

This entire process, from isolation of mRNA to insertion of the cDNA into a vector (e.g., plasmid, viral vector, cosmid, etc.) to growth of host cell populations containing the isolated gene or gene portions, is termed “cDNA cloning.” If cDNAs are prepared from a number of different mRNAs, the resulting set of cDNAs is called a “cDNA library,” an appropriate term since the set of cDNAs represents a “population” of genes or portions of genes comprising the functional genetic information present in the source cell, tissue or organism.

Synthesis of a cDNA molecule initiates at or near the 3′ termini of the mRNA molecules and proceeds in the 5′ to 3′ direction successively adding nucleotides to the growing strand. Priming of cDNA synthesis at the 3′ termini at the poly A tail using an oligo(dT) primer ensures that the 3′ message of the mRNAs will be represented in the cDNA molecules produced. The ability to increase sensitivity and specificity during cDNA synthesis provides more representative cDNA libraries and may increase the likelihood of the cDNA library having full-length cDNA molecules (e.g., full-length genes). Such advances would greatly improve the probability of finding full-length genes of interest.

Therefore, there is a need for a method for improving the ability of polymerases and reverse transcriptases to synthesize nucleic acid molecules. Such advances would provide for improvements in nucleic acid synthesis, sequencing, amplification and cDNA synthesis.

### SUMMARY OF THE INVENTION

The present invention satisfies the need discussed above. The present invention provides a method for inhibiting, reducing, substantially reducing or eliminating nucleic acid synthesis/degradation under certain conditions (preferably at ambient temperatures). In a preferred aspect, the invention prevents or inhibits nucleic acid synthesis and primer degradation during reaction set up and preferably before optimum reaction conditions for nucleic acid synthesis are achieved. Such inhibition of DNA polymerase activities at sub-optimum conditions or during reaction set up prevents or reduces non-specific nucleic acid synthesis. Once reaction set up is complete and the optimum conditions are reached, nucleic acid synthesis can be initiated.

More specifically, the invention relates to controlling nucleic acid synthesis by introducing any polypeptide (preferably a polypeptide having reduced, substantially reduced or no polymerase activity) which binds double-stranded nucleic acids or double-stranded containing nucleic acid molecules such as double-stranded/single-stranded complexes. Such double-stranded nucleic acid molecules may contain single-stranded regions (preferably at one or both termini), or may contain sequences or nucleotides which are not base paired with a complementary nucleic acid strand, or may be completely double-stranded. Accordingly, such polypeptides can bind or interact with such double-stranded nucleic acid molecules (e.g., double-stranded substrates such as a primer/template complex or a double-stranded template) and interfere with nucleic acid synthesis by preventing binding or interaction of an active polymerase or reverse transcriptase with a substrate such as a primer/template complex. In a preferred aspect, the polypeptides of the invention may be preferentially inactivated, substantially reduced or eliminated the binding activity of the polypeptides without inactivating polymerases or reverse transcriptases (or other components) need for nucleic acid synthesis. In one aspect, the polypeptides of the invention are inactivated by heat (temperature change), pH or ionic strength, or other conditions which may be determined by one of ordinary skill in the art.

In another aspect, the invention relates to controlling nucleic acid synthesis by introducing any polypeptide (preferably a polypeptide having reduced, substantially reduced or no nuclease activity (particularly exonuclease activity such as 3′ exonuclease and/or 5′ exonuclease activity)) which binds to nucleic acids, particularly single-stranded or single-stranded containing nucleic acids. Accordingly, such polypeptides can bind to or interact with nucleic acid molecules (e.g., nucleic acid synthesis substrates such as single stranded primers or single stranded templates or double-stranded molecules) and interfere with nucleic acid synthesis, for example, by preventing binding or interaction or hybridization of the nucleic acid synthesis substrates (such as primer with the template to form the primer/template complex substrate used by polymerases or reverse transcriptases in synthesis reactions) or prevent interaction of the polymerase or reverse transcriptase with the synthesis substrates. In addition, the interaction of the polypeptide of the invention with nucleic acid molecules, particularly single-stranded nucleic acids (e.g., single-stranded substrates such as primers and templates) prevents such molecules from being degraded by nucleases (such as exonucleases) that may be present. The polypeptides of the invention thus prevents degradation of substrates used in nucleic acid synthesis, amplification and sequencing reactions, but also prevents degradation of the products produced by such reactions. For example, numerous polymerases used in nucleic acid synthesis, amplification and sequencing have exonuclease activity (e.g., 3′ to 5′ and 5′ to 3′ exonuclease activity of DNA polymerases) which may degrade single-stranded nucleic acid substrates or products and adversely affect the efficiency of nucleic acid synthesis reaction. Moreover, reaction mixtures used in synthesis, amplification and sequencing may contain added nucleases (which may be added to the reaction mixture for a particular purpose or function) or contaminating nucleases (e.g., RNase's, DNase's, and exonucleases and specifically single-stranded exonucleases) which may degrade nucleic acid substrates or products in the reaction mixture. By including the polypeptides to the invention, it is possible to prevent or inhibit degradation of the nucleic acid molecules or substrates (particularly single-stranded molecules or single-strands containing molecules) before or during or after nucleic acid synthesis, amplification and sequencing.

The polypeptides of the invention (which may be referred to as “inhibitory polypeptides”) preferably include enzymes or proteins which bind or interact with any nucleic acid molecules such as double-stranded nucleic acid molecules and/or single-stranded nucleic acid molecules and/or single-stranded/double-stranded nucleic acid complexes and which have been modified or mutated to reduce, substantially reduce or eliminate any polymerase activity and/or nuclease activity, or which naturally have little or no polymerase activity and/or nuclease activity. Examples include transferases, ligases, reverse transcriptases, helicases, topoisomerases, restriction enzymes, DNA repair enzymes, recombination proteins, endonucleases, RNase's (RNase A, RNase T1, RNase H etc.), DNase's (DNase 1, DNase A, etc.) exonucleases (preferably single-stranded specific exonuclease such as epsilon subunit (ε) from pol III type DNA polymerases, 3′ to 5′ and 5′ to 3′ exonucleases from pol I type DNA polymerases, 3′ to 5′ and 5′ to 3′ exonuclease from Family A type DNA polymerases, 3′ to 5′ exonuclease from Family B type DNA polymerases and 3′ to 5′ and 5′ to 3′ exonuclease subunits from Family C type DNA polymerases) and polymerases (preferably mesophilic polymerases). Preferred examples include any wild-type or mutant polymerase or reverse transcriptase having double-stranded nucleic acid binding activity with reduced, substantially reduced, or no polymerase activity and optionally reduced, substantially reduced or no exonuclease activity. Preferred examples also include wild-type or mutant exonucleases (or other enzymes having exonuclease activity such as 3′ exonuclease and/or 5′ exonuclease found in DNA polymerases) which have nucleic acid (double-stranded and preferably, single-stranded) binding activity with reduced substantially reduced, or no exonuclease activity.

In a preferred aspect, the polypeptides of the invention are modified or mutated to reduce, substantially reduce or eliminate or naturally have little or no exonuclease activity and polymerase activity. Thus, in a preferred aspect, the polypeptides are capable of binding one or more double-stranded nucleic acid substrates and one or more single-stranded nucleic acid substrates, but since they possess little or no polymerase activity and little or no exonuclease activity (e.g. 3′ to 5′ and/or 5′ to 3′ exonuclease activity), little or no synthesis of a nucleic acid molecule complementary to all or a portion of the template will occur. Additionally, little or no degradation of nucleic acid molecules in the reaction mixture will occur. Thus, the polypeptide is preferably introduced into the reaction mixture where it competitively binds to or interacts with the substrate(s) (e.g., primer/template complexes, double stranded molecules and/or single-stranded molecules such as single-stranded primers and single stranded templates), thereby inhibiting nucleic acid synthesis in the presence of one or more enzymes having polymerase or reverse transcriptase activity under particular reaction conditions. The polypeptides of the invention also have the ability to interact or bind with the synthesized products and/or substrates of the reaction mixture, thereby preventing degradation of the products or substrates with nucleases which may be present in the reaction mixture.

In another aspect, the polypeptides in the invention are modified or mutated nucleases having reduced, substantially reduced or eliminated nuclease activity. Preferred nucleases (preferably thermolabile or mesophilic nucleases) in this aspect of the invention are exonucleases and particularly single-stranded specific exonucleases. Such nucleases naturally interact or bind nucleic acids and the modifications and mutations preferably should have little or no adverse affect on the ability of the nuclease to bind nucleic acids (although modification or mutations may be incorporated to enhance such binding/interaction activity). Thus, in a preferred aspect, one or more exonucleases which are preferable single-stranded specific exonucleases are modified or mutated and thus are capable binding one or more nucleic acid substrates but since they possess little or no exonuclease activity, they are capable of preventing synthesis with such substrates (e.g., single-stranded templates and single-stranded primers). Such synthesis is prevented, for example, by preventing interaction of the nucleic acids with active polymerases/reverse transcriptases and/or by preventing interaction of the nucleic acid molecules (such as hybridization to form primer/template complexes). Such polypeptide also prevent degradation of nucleic acid molecules in the reaction since they bind such molecules, preferably making them inaccessible to the action of other nucleases. Thus, such polypeptide is preferably introduced into a reaction mixture where it competitively binds to or interacts with such nucleic acid molecules, thereby inhibiting nucleic acid synthesis and nucleic acid degradation in the presents of one or more enzymes having polymerase and/or nuclease activity.

In another aspect, the polypeptides of the invention are modified or mutated polymerases having reduced, substantially reduced or eliminated polymerase activity. Preferred polymerases in this aspect are DNA polymerases and reverse transcriptases and particularly thermolabile or mesophilic DNA polymerases and reverse transcriptases. Such polymerases naturally interact or bind nucleic acid (preferably nucleic acid substrates used in nucleic acid synthesis such as double-stranded molecule having one or more single-stranded regions preferably at one or both termini, for example, primers/template complexes) and the modifications and mutations preferably should have little or no adverse effect on the ability of the polymerase to bind nucleic acids (although modifications or mutations may be incorporated to enhance such binding/interaction activity). Such polypeptides are capable of binding one or more nucleic acid substrates but since they possess little or no polymerase activity, they bind to or interact with such nucleic acid substrates (e.g., a primer/template complex) needed for nucleic acid synthesis. Thus, the polypeptide is preferable introduced into a reaction mixture where it competitively binds to or interacts with such substrates, thereby inhibiting nucleic acid synthesis in the presence of one or more enzymes having polymerase activity. Such synthesis is prevented, for example, by preventing interaction of the nucleic acids with active polymerases/reverse transcriptases and/or by preventing interaction of the nucleic acid molecules (such as hybridization to form primer/template complexes).

The inhibition of nucleic acid synthesis or the interaction/binding by the polypeptides of the invention is preferably eliminated or reduced so that nucleic acid synthesis may proceed when reaction conditions are changed, for example, when the temperature is raised. In a preferred aspect, the changed conditions affect the ability of the polypeptides to interact with double-stranded nucleic acid substrates and/or single-stranded nucleic acid substrates and/or single-stranded/double-stranded complexes, causing release of the substrates and/or denaturation or inactivation of the polypeptides making the nucleic acid molecules available as substrates for the enzyme with polymerase/reverse transcriptase activity thus allowing nucleic acid synthesis to proceed.

The invention therefore relates to a method for synthesizing one or more nucleic acid molecules, comprising (a) mixing one or more nucleic acid templates (which may be a DNA molecule such as a cDNA molecule, or an RNA molecule such as a mRNA molecule) with one or more primers, and one or more polypeptides or compositions of the present invention capable of binding or interacting with one or more double-stranded and/or single-stranded nucleic acid substrates and/or single-stranded/double-stranded complexes (e.g., substrates for nucleic acid synthesis such as templates, template/primer complexes and/or primers) wherein said polypeptide has reduced, substantially reduced, or no polymerase activity and/or reduced, substantially reduced, or no nuclease activities and (b) incubating the mixture in the presence of one or more enzymes having nucleic acid polymerase activity and/or nuclease activity (e.g., DNA polymerases and/or reverse transcriptases and/or nucleases such as endonucleases and exonucleases) under conditions sufficient to synthesize one or more first nucleic acid molecules complementary to all or a portion of the templates. Such mixing is preferably accomplished under conditions to prevent nucleic acid synthesis and/or to allow binding of the polypeptide of the invention to one or more nucleic acid synthesis substrates. In a preferred aspect, the synthesis conditions are sufficient to inactivate or denature the polypeptide of the invention to inhibit, reduce, substantially reduce or eliminate binding of said polypeptide to the nucleic acid synthesis substrates. Such incubation conditions may involve the use of one or more nucleotides and one or more nucleic acid synthesis buffers. Preferably, the incubation conditions are accomplished at a temperature sufficient to inactivate the polypeptides of the invention and/or prevent binding of the polypeptides to the nucleic acid synthesis substrates, but at a temperature insufficient to inactivate the polymerases and/or reverse transcriptases or other enzymes present and needed for the nucleic acid synthesis reaction. Such methods of the invention may optionally comprise one or more additional steps, such as incubating the synthesized first nucleic acid molecules under conditions sufficient to make one or more second nucleic acid molecules complementary to all or a portion of the first nucleic acid molecules. Such additional steps may also be accomplished in the presence of the polypeptides/compositions of the invention as described herein. The invention also relates to nucleic acid molecules synthesized by this method.

More specifically, the invention relates to a method of amplifying a DNA molecule comprising: (a) mixing a first and second primer, wherein said first primer is complementary to a sequence at or near the 3′-termini of the first strand of said DNA molecule and said second primer is complementary to a sequence at or near the 3′-termini of the second strand of said DNA molecule and one or more polypeptides or compositions of the invention (e.g., a polypeptide with affinity to double-stranded nucleic acids and/or single-stranded nucleic acids and/or single-stranded/double-stranded complexes and having reduced, substantially reduced, or no polymerase activity and/or nuclease activity); (b) hybridizing said first primer to said first strand and said second primer to said second strand; (c) incubating the mixture under conditions such that a third DNA molecule complementary to all or a portion of said first strand and a fourth DNA molecule complementary to all or a portion of said second strand are synthesized; (d) denaturing said first and third strand, and said second and fourth strands; and (e) repeating steps (a) to (c) or (d) one or more times. Such mixing is preferably accomplished under conditions to prevent nucleic acid synthesis and/or to allow binding of the polypeptide of the invention to one or more nucleic acid synthesis substrates. In a preferred aspect, the synthesis conditions are sufficient to inactivate or denature the ability of the polypeptide of the invention to inhibit, reduce, substantially reduce or eliminate binding of said polypeptide to the nucleic acid synthesis substrates. Preferably, the incubation conditions are accomplished at a temperature sufficient to inactivate the polypeptides of the invention and/or prevent binding of the polypeptides to the nucleic acid synthesis substrates, but at a temperature insufficient to inactivate the polymerases and/or reverse transcriptases or other enzymes present and needed for the nucleic acid synthesis reaction. Such incubation conditions may include incubation in the presence of one or more polymerases, one or more nucleotides and/or one or more buffering salts. The invention also relates to nucleic acid molecules amplified by these methods.

The invention also relates to methods for sequencing a nucleic acid molecule comprising (a) mixing a nucleic acid molecule to be sequenced with one or more primers, one or more of the polypeptides or compositions of the invention, one or more nucleotides and one or more terminating agents to form a mixture; (b) incubating the mixture under conditions sufficient to synthesize a population of molecules complementary to all or a portion of the molecule to be sequenced; and (c) separating the population to determine the nucleotide sequence of all or a portion of the molecule to be sequenced. The invention more specifically relates to a method of sequencing a nucleic acid molecule, comprising: (a) mixing a polypeptide or composition of the present invention (having affinity to double-stranded nucleic acids and/or single stranded nucleic acids and/or single-stranded/double-stranded complexes and having reduced, substantially reduced, or no polymerase activity and/or nuclease activity), one or more nucleotides, and one or more terminating agents; (b) hybridizing a primer to a first nucleic acid molecule; (c) incubating the mixture of step (b) under conditions sufficient to synthesize a random population of nucleic acid molecules complementary to said first nucleic acid molecule, wherein said synthesized molecules are shorter in length than said first molecule and wherein said synthesized molecules comprise a terminator nucleotide at their 3′ termini; and (d) separating said synthesized molecules by size so that at least a part of the nucleotide sequence of said first nucleic acid molecule can be determined. Such mixing is preferably accomplished under conditions to prevent nucleic acid synthesis and/or to allow binding of the polypeptide of the invention to one or more nucleic acid synthesis substrates. In a preferred aspect, the synthesis conditions and/or hybridization conditions are sufficient to inactivate or denature the polypeptide of the invention to inhibit, reduce, substantially reduce or eliminate binding of said polypeptide to the nucleic acid synthesis substrates. Preferably, the incubation conditions are accomplished at a temperature sufficient to inactivate the polypeptides of the invention and/or prevent binding of the polypeptides to the nucleic acid synthesis substrates, but at a temperature insufficient to inactivate the polymerases and/or reverse transcriptases or other enzymes present and needed for the nucleic acid synthesis reaction. Such terminator nucleotides include ddNTP, ddATP, ddGTP, ddITP or ddCTP. Such incubation conditions may include incubation in the presence of one or more polymerases and/or buffering salts.

The invention also generally relates to methods of preventing or inhibiting the degradation of nucleic acid molecules. Preferably, such methods are preferably preformed in a reaction or reaction mixture during nucleic acid synthesis, cDNA synthesis, amplification or sequencing. Specifically, the methods may comprise: (a) obtaining one or more modified or mutated nucleases having reduced, substantially reduced or no nuclease activity (preferably RNase's, DNase's, and exonucleases and more preferably single-strand specific exonucleases), and (b) contacting said nucleases with one or more nucleic acid molecules under conditions sufficient to prevent degradation of said molecules with one or more nucleases having nuclease activity. The modified or mutated nucleases have affinity for and thus may bind or interact with nucleic acid molecules depending on the specificity of the particular nuclease used. Accordingly, since the nucleases of the invention have been modified to reduce, substantially reduce or eliminate nuclease activity, they are capable of binding nucleic acids and thus preventing interaction or binding of other nucleases with such nucleic acid molecules. In a preferred aspect, the methods of protecting nucleic acid molecules according to the invention are accomplished during in vitro reactions, particularly those reactions used in standard molecular biology techniques (such as nucleic acid synthesis, amplification, sequencing and cDNA synthesis). The degradation protection method of the invention may further comprise the step of inactivating the polypeptide of the invention and/or preventing binding of the polypeptide to the nucleic acid molecules under particular conditions, for example, by heat inactivation of the polypeptides of the invention.

The invention also relates to the polypeptides of the invention and to compositions comprising the polypeptides of the invention, as well as nucleic acid molecules encoding the polypeptides of the present invention, to vectors (which may be expression vectors) comprising these nucleic acid molecules, and to host cells comprising these nucleic acid molecules or vectors. The invention also relates to methods of producing a polypeptide, comprising culturing the above-described host cells under conditions favoring the production of the polypeptide by the host cells, and isolating the polypeptide. The invention also relates to polypeptides produced by such methods.

The invention also relates to kits for use in synthesis, sequencing and amplification of nucleic acid molecules, comprising one or more containers containing one or more of the polypeptides or compositions of the invention. These kits of the invention may optionally comprise one or more additional components selected from the group consisting of one or more nucleotides, one or more templates, one or more polymerases (e.g., thermophilic or mesophilic DNA polymerases) and/or reverse transcriptases, one or more suitable buffers, one or more primers, one or more terminating agents (such as one or more dideoxynucleotides), and instructions for carrying out the methods of the invention. The invention also relates to kits for use in the general methods of preventing or inhibiting degradation of nucleic acid molecules according to the invention. Such kits may comprise one or more containers containing one or more of the polypeptides for compositions in the invention. These kits may optionally comprise one or more additional components selected from the group consisting of one or more nucleotides, one or more templates, one or more polymerases (e.g., thermophilic or mesophilic DNA polymerases) and/or reverse transcriptases, one or more suitable buffers, one or more primers, one or more terminating agents, and instructions for carrying out this method of the invention.

The invention also relates to compositions for use in synthesis, sequencing and amplification of nucleic acid molecules and to compositions made for carrying out such synthesis, sequencing and amplification reactions. The invention also relates to compositions made during or after carrying out the synthesis, sequencing and amplification reactions of the invention. Such compositions of the invention may comprise one or more of the inhibitory polypeptides of the invention and may further comprise one or more components selected from the group consisting of one or more nucleotides, one or more primers, one or more templates, one or more reverse transcriptases, one or more DNA polymerases, one or more buffers, one or more buffer salts and one or more synthesized nucleic acid molecules made according to the methods of the invention. The invention also relates to the compositions for use in the methods of preventing or inhibiting degradation in nucleic acid molecules and to compositions made for carrying out such methods. The invention also relates to compositions made during or after carrying out such methods of protecting against degradation in nucleic acid molecules. Such compositions of the invention may comprise one or more of the inhibitory polypeptides of the invention and may further comprise one or more components selected from the group consisting of one or more nucleotides, one or more primers, one or more templates, one or more reverse transcriptases, one or more polymerases (DNA polymerases and reverse transcriptases), one or more buffers, one or more buffering salts, and one or more synthesized nucleic acid molecules made according to this method of the invention.

Other preferred embodiments of the present invention will be apparent to one of ordinary skill in light of the following drawings and description of the invention, and of the claims.

### BRIEF DESCRIPTION OF THE DRAWINGS

FIG. 1 shows inhibition of DNA polymerization reaction catalyzed by a reverse transcriptase (RT) using an inactivated Klenow fragment (pol⁻ and exo⁻) derivative of polymerase I of E. coli at ambient temperature. P denotes the position of the DNA primer (34-mer) and F.L. is the fully extended product (60-mer). Panels A and B indicate DNA polymerase reactions catalyzed by Thermoscript™ (RNase H deficient mutant of reverse transcriptase) as a function of the concentration of the Klenow fragment derivative at ambient temperature and 50° C., respectively. The Klenow fragment:RT ratio in the reaction mix were—for lanes denoted as: a, Klenow fragment was not added; b, 52:1; c, 26:1; d, 5.2:1 and e, 1:1. For each protein condition the reaction was stopped after 1 and 6 min of incubation.

Residual polymerase activity of the mutant derivative of the Klenow fragment used for the inhibition polymerase activity shown in panels A & B is shown in panel C. Three time points (1, 5 & 20 min from left to right) denotes the polymerase reaction catalyzed by the mutant Klenow fragment, carrying the mutations K758A & D882A (pol⁻) and D355A and E357A (exo⁻).

FIG. 2 shows inhibition of DNA polymerization reaction catalyzed by Taq, Tne (5′ to 3′ exo⁻; D137A) and KOD thermophilic DNA polymerases by an inactivated Klenow fragment (pol⁻ and exo⁻) derivative of polymerase I of E. coli at ambient temperature. P denotes the position of the DNA substrate (primer) and F.L. is the fully extended product. Lanes labeled a, b, and c indicate reaction temperatures at ambient temperature, 55° C. and 72° C., respectively. For reactions at ambient temperature and 55° C. the reaction was stopped at 30 sec and 2 min whereas at 72° C. it was only stopped at 30 sec after initiation of polymerization. For each of the three polymerase, the left panels are for polymerization catalyzed in the presence of excess Klenow fragment, whereas the right panels are for reactions catalyzed in the absence of Klenow fragment.

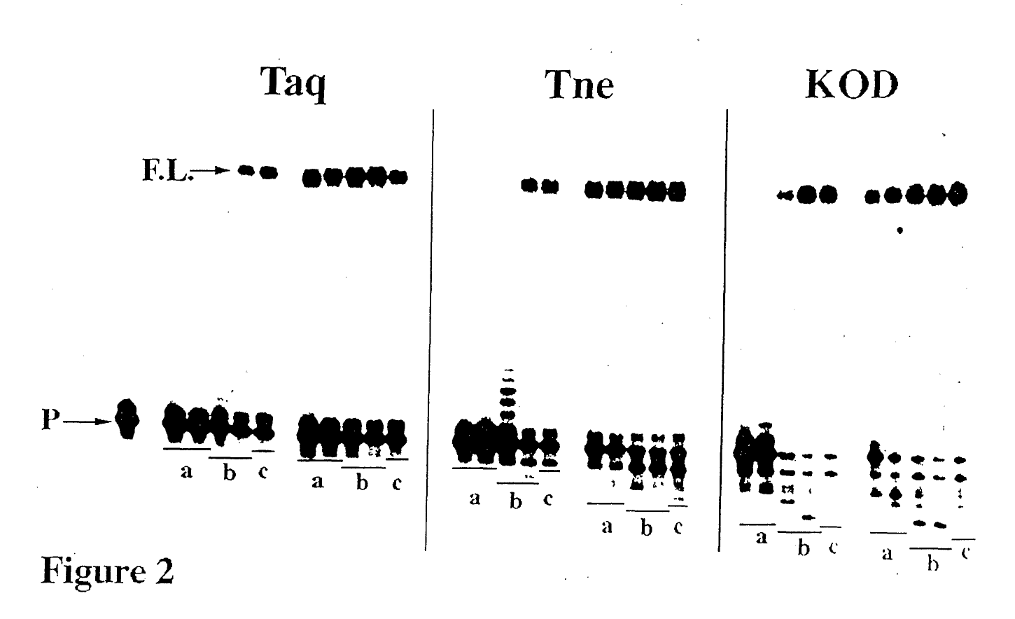

FIG. 3 shows inhibition of degradation of single-stranded primers with a mutant Klenow fragment ((pol⁻ and exo⁻) in the presence of Tne (pol⁺, 3′ to 5′ exonuclease⁺ and 5′ to 3′ exonuclease⁻). FIG. 3 shows the inhibition of the 3′ to 5′ exonuclease reaction catalyzed by Tne DNA polymerase (5′exo⁻/D137A) using an inactivated Klenow fragment derivative (K758A, D882A, D355A and E357A) of polymerase I of E. coli at ambient temperature and 37° C. P denotes the position of the DNA substrate (34-mer). Lane C (left lane) is a control lane of the labeled oligonucleotide substrate. Panels A, B, C and D indicate the 3′ to 5′ exonuclease reactions catalyzed by Tne DNA polymerase at varying concentrations of the Klenow fragment. Panel A represent the reaction in the absence of Klenow fragment; Panels B, C and D represent reactions in the presence of 5 μM, 10 μM and 20 μM of Klenow fragment, respectively. For each reaction condition the DNA substrate and Tne DNA polymerase concentrations were maintained at 9 nM and 60 nM, respectively. The exonuclease digestion of the 34-mer substrate was measured at ambient temperature, 37° C. and 72° C. For each reaction condition the digestion was stopped at 5 and 20 min following the initiation of the reaction by the addition of Tne. The left and right lanes of each temperature reaction sub-panels represent reactions quenched at 5 and 20 min.

### DETAILED DESCRIPTION OF THE INVENTION

Definitions

In the description that follows, a number of terms used in recombinant DNA technology are utilized extensively. In order to provide a clearer and consistent understanding of the specification and claims, including the scope to be given such terms, the following definitions are provided.

Primer. As used herein, “primer” refers to a single-stranded oligonucleotide that is extended by covalent bonding of nucleotide monomers during amplification or polymerization of a nucleic acid molecule.

Template. The term “template” as used herein refers to double-stranded or single-stranded nucleic acid molecules (RNA and/or DNA) which are to be amplified, synthesized or sequenced. In the case of a double-stranded molecules, denaturation of its strands to form a first and a second strand is preferably performed before these molecules may be amplified, synthesized or sequenced, or the double-stranded molecule may be used directly as a template. For single stranded templates, a primer, complementary to a portion of the template is hybridized under appropriate conditions and one or more polymerases may then synthesize a nucleic acid molecule complementary to all or a portion of said template. Alternatively, for double-stranded templates, one or more promoters (e.g. SP6, T7 or T3 promoters) may be used in combination with one or more polymerases to make nucleic acid molecules complementary to all or a portion of the template. The newly synthesized molecules, according to the invention, may be equal or shorter in length than the original template.

Incorporating. The term “incorporating” as used herein means becoming a part of a DNA and/or RNA molecule or primer.

Amplification. As used herein “amplification” refers to any in vitro method for increasing the number of copies of a nucleotide sequence with the use of a polymerase. Nucleic acid amplification results in the incorporation of nucleotides into a DNA and/or RNA molecule or primer thereby forming a new molecule complementary to all or a portion of a template. The formed nucleic acid molecule and its template can be used as templates to synthesize additional nucleic acid molecules. As used herein, one amplification reaction may consist of many rounds of replication. DNA amplification reactions include, for example, polymerase chain reactions (PCR). One PCR reaction may consist of 5 to 100 “cycles” of denaturation and synthesis of a DNA molecule.

Nucleotide. As used herein “nucleotide” refers to a base-sugar-phosphate combination. Nucleotides are monomeric units of a nucleic acid sequence (DNA and RNA). The term nucleotide includes ribonucleoside triphosphates ATP, UTP, CTG, GTP and deoxyribonucleoside triphosphates such as dATP, dCTP, dITP, dUTP, dGTP, dTTP, or derivatives thereof. Such derivatives include, for example, [αS]dATP, 7-deaza-dGTP and 7-deaza-dATP, and nucleotide derivatives that confer nuclease resistance on the nucleic acid molecule containing them. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP. According to the present invention, a “nucleotide” may be unlabeled or detectably labeled by well known techniques. Detectable labels include, for example, radioactive isotopes, fluorescent labels, chemiluminescent labels, bioluminescent labels and enzyme labels.

Oligonucleotide. “Oligonucleotide” refers to a synthetic or natural molecule comprising a covalently linked sequence of nucleotides which are joined by a phosphodiester bond between the 3′ position of the deoxyribose or ribose of one nucleotide and the 5′ position of the deoxyribose or ribose of the adjacent nucleotide.

Hybridization. The terms “hybridization” and “hybridizing” refers to base pairing of two complementary single-stranded nucleic acid molecules (RNA and/or DNA) to give a double-stranded molecule. As used herein, two nucleic acid molecules may be hybridized, although the base pairing is not completely complementary. Accordingly, mismatched bases do not prevent hybridization of two nucleic acid molecules provided that appropriate conditions, well known in the art, are used.

Unit. The term “unit” as used herein refers to the activity of an enzyme. When referring, for example, to a DNA polymerase, one unit of activity is the amount of enzyme that will incorporate 10 nanomoles of dNTPs into acid-insoluble material (i.e., DNA or RNA) in 30 minutes under standard primed DNA synthesis conditions.

Vector. A plasmid, phagemid, cosmid or phage DNA or other DNA molecule which is able to replicate autonomously in a host cell, and which is characterized by one or a small number of restriction endonuclease recognition sites at which such DNA sequences may be cut in a determinable fashion without loss of an essential biological function of the vector, and into which DNA may be spliced in order to bring about its replication and cloning. The cloning vector may further contain a marker suitable for use in the identification of cells transformed with the cloning vector. Markers, for example, are tetracycline resistance or ampicillin resistance.

Expression vector. A vector similar to a cloning vector but which is capable of enhancing the expression of a gene which has been cloned into it, after transformation into a host. The cloned gene is usually placed under the control of (i.e., operably linked to) certain control sequences such as promoter sequences.

Recombinant host. Any prokaryotic or eukaryotic microorganism which contains the desired cloned genes in an expression vector, cloning vector or any DNA molecule. The term “recombinant host” is also meant to include those host cells which have been genetically engineered to contain the desired gene on the host chromosome or genome.

Host. Any prokaryotic or eukaryotic microorganism that is the recipient of a replicable expression vector, cloning vector or any DNA molecule. The DNA molecule may contain, but is not limited to, a structural gene, a promoter and/or an origin of replication.

Promoter. A DNA sequence generally described as the 5′ region of a gene, located proximal to the start codon. At the promoter region, transcription of an adjacent gene(s) is initiated.

Gene. A DNA sequence that contains information necessary for expression of a polypeptide or protein. It includes the promoter and the structural gene as well as other sequences involved in expression of the protein.

Structural gene. A DNA sequence that is transcribed into messenger RNA that is then translated into a sequence of amino acids characteristic of a specific polypeptide.

Operably linked. As used herein means that the promoter is positioned to control the initiation of expression of the polypeptide encoded by the structural gene.

Expression. Expression is the process by which a gene produces a polypeptide. It includes transcription of the gene into messenger RNA (mRNA) and the translation of such mRNA into polypeptide(s).

Substantially Pure. As used herein “substantially pure” means that the desired purified protein or polypeptide is essentially free from contaminating cellular contaminants which are associated with the desired protein or polypeptide in nature. Contaminating cellular components may include, but are not limited to, phosphatases, exonucleases, endonucleases or undesirable DNA polymerase enzymes.

Thermostable. As used herein “thermostable” refers to a polypeptide having polymerase activity (e.g. DNA polymerase and reverse transcriptase) which is resistant to inactivation by heat. By way of example, DNA polymerases synthesize the formation of a DNA molecule complementary to a single-stranded DNA template by extending a primer in the 5′ to 3′ direction. This activity for mesophilic DNA polymerases may be inactivated by heat treatment. For example, T5 DNA polymerase activity is totally inactivated by exposing the enzyme to a temperature of 90° C. for 30 seconds. As used herein, a thermostable polymerase activity is more resistant to heat inactivation than a mesophilic polymerase. However, a thermostable polymerase does not mean to refer to an enzyme which is totally resistant to heat inactivation and thus heat treatment may reduce the polymerase activity to some extent. A thermostable polymerase typically will also have a higher optimum temperature than mesophilic polymerases.

3′ to 5′ Exonuclease Activity. “3′ to 5′ exonuclease activity” is an enzymatic activity well known to the art. This activity is often associated with DNA polymerases, and is thought to be involved in a DNA replication “editing” or correction mechanism.

A “polymerase substantially reduced in 3′ to 5′ exonuclease activity” is defined herein as either (1) a mutated or modified polymerase that has about or less than 10%, or preferably about or less than 1%, of the 3′ to 5′ exonuclease activity of the corresponding unmutated, wild-type enzyme, or (2) a polymerase having a 3′ to 5′ exonuclease specific activity which is less than about 1 unit/mg protein, or preferably about or less than 0.1 units/mg protein. A unit of activity of 3′ to 5′ exonuclease is defined as the amount of activity that solubilizes 10 nmoles of substrate ends in 60 min. at 37° C., assayed as described in the “BRL 1989 Catalogue & Reference Guide”, page 5, with Hhal fragments of lambda DNA 3′-end labeled with [³H]dTTP by terminal deoxynucleotidyl transferase (TdT). Protein is measured by the method of Bradford, Anal. Biochem. 72:248 (1976). As a means of comparison, natural, wild-type T5-DNA polymerase (DNAP) or T5-DNAP encoded by pTTQ19-T5-2 has a specific activity of about 10 units/mg protein while the DNA polymerase encoded by pTTQ19-T5-2(Exo-) (U.S. Pat. No. 5,270,179) has a specific activity of about 0.0001 units/mg protein, or 0.001% of the specific activity of the unmodified enzyme, a 105-fold reduction.

5′ to 3′ Exonuclease Activity. “5′ to 3′ exonuclease activity” is also an enzymatic activity well known in the art. This activity is often associated with DNA polymerases, such as E. coli PolI and Taq DNA polymerase.

A “polymerase substantially reduced in 5′ to 3′ exonuclease activity” is defined herein as either (1) a mutated or modified polymerase that has about or less than 10%, or preferably about or less than 1%, of the 5′ to 3′ exonuclease activity of the corresponding unmutated, wild-type enzyme, or (2) a polymerase having 5′ to 3′ exonuclease specific activity which is less than about 1 unit mg protein, or preferably about or less than 0.1 units/mg protein.

Both of the 3′ to 5′ and 5′ to 3′ exonuclease activities can be observed on sequencing gels. Active 5′ to 3′ exonuclease activity will produce nonspecific ladders in a sequencing gel by removing nucleotides from the 5′-end of the growing primers. 3′ to 5′ exonuclease activity can be measured by following the degradation of radiolabeled primers in a sequencing gel. Thus, the relative amounts of these activities, e.g., by comparing wild-type and mutant or modified polymerases, can be determined with no more than routine experimentation.

Reduced nuclease activity. Polypeptides with reduced nuclease activity include nucleases (DNase's, RNase's endonucleases, exonucleases etc.) wherein the ability to degrade nucleic acid molecules (such as single-stranded and double-stranded nucleic acid molecules) has been reduced. Preferred are exonucleases having reduced activity such as single-strand specific exonucleases, although endonucleases are contemplated by the invention. Nuclease activity of a polypeptide can be reduced by any means including chemical or physical treatment or modification, such as temperature (e.g., heat inactivation), ionic strength (salt or pH), enzymatic treatment (proteinases), and genetic modification and mutations. Genetic modification or mutation are preferably accomplished by introducing mutations or modifications into the nucleic acid molecule (gene or genes) encoding the nuclease of interest by well known techniques such that expression of the nucleic acid results in an nuclease with reduced nuclease activity. See Monk, M. and Kinross J., J. Bacteriol. 109, 971-978, 1972 and Kingbury, D. and Helinsi, D., J. Bacteriology 114, 1116, 1124, 1973. Preferably, the nuclease activity is reduced by at least 30%, more preferably reduced at least about 50%, and most preferably reduced at least more than about 75% compared to the corresponding untreated or unmodified exonuclease. Such modifications and mutations may include point mutations, substitutions, and deletion mutations (or combinations thereof) made by well known techniques. Furthermore, assays described herein and known in the art for determining the level or nuclease activity can be used to select desired clones having reduced nuclease activity.

Other mutations may be introduced into the nucleases of the invention to enhance function in a desired way, for example its affinity for single-stranded nucleic acids or other nucleic acid molecules, its temperature sensitivity (e.g., to lower the temperature needed to inhibit or prevent binding or interaction of the nucleases of the invention to single-stranded nucleic acid molecules or other nucleic acid molecules such as single-stranded primers or other nucleic acid molecules).

Substantially reduced nuclease activity. A polypeptide with substantially reduced nuclease activity is defined herein as any nuclease that has about or less than 20%, more preferably about or less than 15%, still more preferably about or less than 10%, and most preferably about or less than 1%, of the nuclease activity of the corresponding unmutated, unmodified or wild-type enzyme. Modifications or mutations to create such polypeptides may include point mutations, substitutions and deletion mutations (or combinations thereof) made by well known techniques.

Reduced polymerase activity. Polypeptides with reduced polymerase activity include polymerases or reverse transcriptases wherein the ability to synthesize the formation of a nucleic acid molecule complementary to a single-stranded nucleic acid template has been reduced. Polymerase activity of a polypeptide can be reduced by any means including chemical or physical treatment or modification, such as temperature (e.g., heat inactivation), ionic strength (salt or pH), enzymatic treatment (proteinases), and genetic modification or mutations. Genetic modification or mutation is preferably accomplished by introducing mutations or modifications into the nucleic acid molecule (gene or genes) encoding the polypeptide or polymerase of interest by well known techniques such that expression of the nucleic acid results in a polymerase or polypeptide with reduced polymerase activity. See Monk, M. and Kinross J., J. Bacteriol. 109, 971-978, 1972 and Kingbury, D. and Helinsi, D., J. Bacteriology 114, 1116, 1124, 1973. Preferably, the polymerase activity is reduced by at least about 30%, more preferably reduced at least about 50%, and most preferably reduced at least more than about 75% compared to the untreated or unmodified polypeptide. Such modifications or mutations may include point mutations, substitutions, and deletion mutations (or combinations thereof) made by well known techniques. Furthermore, assays described herein and known in the art for determining the level of polymerase activity can be used to select desired clones having reduced polymerase activity.

Other mutation may be introduced into the polypeptides of the invention to enhance function in a desired way, for example its affinity for double-stranded nucleic acids, its temperature sensitivity (e.g. to lower the temperature needed to inhibit or prevent binding or interaction of the polypeptide to the double-stranded nucleic acid molecules such as the primer/template), or for reducing the exonuclease activity of the polymerase (e.g. 3′ to 5′ and/or 5′ to 3′ exonuclease activity). For example, the mutation G522D provides a temperature sensitive Pol I DNA polymerase. Such a mutant polymerase may be inactivated or denatured at a temperature at or below 37° C. Corresponding mutations may be made in any other protein or enzyme (such as a reverse transcriptase or polymerase) to provide for a temperature sensitive protein or enzyme which binds double-stranded nucleic acid molecules for use in the invention.

Substantially reduced polymerase activity. A polypeptide with substantially reduced polymerase activity is defined herein as any polypeptide (e.g., polymerase or reverse transcriptase) that has about or less than about 25%, more preferably about or less than 20%, more preferably about or less than 15%, still more preferably about or less than 10%, and most preferably about or less than 1%, of the polymerase activity of the corresponding unmutated, unmodified or wild-type enzyme. Modifications or mutations to create such polypeptides may include point mutations, substitutions, and deletion mutations (or combinations thereof) made by well known techniques.

As described above, other mutations may be introduced into the polypeptides of the invention to enhance function in a desired way, for example its affinity to double-stranded nucleic acids, its temperature sensitivity (e.g. to lower the temperature needed to inhibit or prevent binding of the polypeptide to the template), or for reducing the exonuclease activity of the polymerase (e.g. 3′ to 5′ and/or 5′ to 3′ exonuclease activity). Furthermore, the polymerase activity of a mutated or modified polypeptide can be determined by the methods described herebelow or any other method known in the art. A polypeptide with substantially reduced polymerase may still bind double-stranded nucleic acids.

Other terms used in the fields of recombinant DNA technology and molecular and cell biology as used herein will be generally understood by one of ordinary skill in the applicable arts.

Inhibitory Polypeptides

The polypeptides of the present invention include a variety of polypeptides (including proteins and enzymes) having affinity for double-stranded nucleic acids i.e. DNA/DNA, DNA/RNA, RNA/RNA, PNA/DNA, PNA/RNA, LNA/DNA or LNA/RNA and/or for single-stranded nucleic acids (e.g., RNA or DNA or PNA or LNA) and/or single-stranded/double-stranded nucleic acid complexes (or combinations thereof). Such polypeptides may be derived from any proteins or enzymes which bind to or have affinity for such nucleic acid molecules. Examples of such proteins and/or enzymes include but are not limited to ligases, polymerases (DNA and RNA polymerases), restriction endonucleases, exonucleases, nucleases (e.g., single-stranded specific and double-stranded nucleases), endonucleases, DNase's, RNase's, reverse transcriptase, transcription factors, topoisomerases, DNA repair enzymes (mutL, mutS, etc.), recombination proteins (Int, resolvase, Cre, X is, Flp, etc.), DNA replication enzymes (helicases and methylases) and the like. As will be recognized, other polypeptides (natural, unnatural, modified etc.) may be selected and used in accordance with the invention. Such selection may be accomplished by double-stranded and/or single-stranded and/or single-stranded/double-stranded nucleic acid complex nucleic acid binding studies and/or nucleic acid synthesis inhibition assays. Preferred proteins and enzymes used in deriving the polypeptides of the invention include polymerases or reverse transcriptases or nucleases (particularly exonucleases). In such case where a polymerase or reverse transcriptase is used, the protein or enzyme is preferably modified or mutated to reduce, substantially reduce or eliminate the polymerase activity of such proteins or enzymes. On the other hand, if the protein or enzyme used naturally has little or no polymerase activity, such modification or mutation may be unnecessary. Polymerases having exonuclease activity domains are preferably modified or mutated to reduce, substantially reduce or eliminate such exonuclease activity (5′ to 3′ and/or 3′ to 5′ exonuclease activity. In such case where a nuclease is used, the protein or enzyme is preferably modified or mutated to reduce, substantially reduce or eliminate the nuclease activity of such proteins or enzymes. On the other hand, if the protein or enzyme used naturally has little or no nuclease activity, such modification or mutation may be unnecessary.

DNA polymerases used to derive the polypeptides and compositions of the invention include, but are not limited to, Thermus thermophilus (Tth) DNA polymerase, Thermus aquaticus (Taq) DNA polymerase, Thermotoga neopolitana (Tne) DNA polymerase, Thermotoga maritima (Tma) DNA polymerase, Thermococcus litoralis (Tli or VENT™) DNA polymerase, Pyrococcus furiosus (Pfu) DNA polymerase, DEEPVENT™ DNA polymerase, Pyrococcus woosii (Pwo) DNA polymerase, Pyrococcus sp KOD2 (KOD) DNA polymerase, Bacillus sterothermophilus (Bst) DNA polymerase, Bacillus caldophilus (Bca) DNA polymerase, Sulfolobus acidocaldarius (Sac) DNA polymerase, Thermoplasma acidophilum (Tac) DNA polymerase, Thermus flavus (Tfl/Tub) DNA polymerase, Thermus ruber (Tru) DNA polymerase, Thermus brockianus (DYNAZYME™) DNA polymerase, Methanobacterium thermoautotrophicum (Mth) DNA polymerase, mycobacterium DNA polymerase (Mtb, Mlep), E. coli pol I DNA polymerase, T5 DNA polymerase, T7 DNA polymerase, and generally pol I, pol III, Family A, Family B and Family C type DNA polymerase and mutants, variants and derivatives thereof. RNA polymerases such as T3, T5 and SP6 and mutants, variants and derivatives thereof may also be used in accordance with the invention. It is preferred that any of the polymerases listed above be modified such that they possess little or no polymerase and optionally little or not exonuclease activity. Mutations which increase DNA affinity have been described Polesky et al., 1990, J. Biol. Chem. 265, 14579-14591. It would be within the skill of a person in the art to alter the polypeptides described above for a desired purpose.

The nucleic acid polymerases used in the present invention may be mesophilic or thermophilic, and are preferably mesophilic. Preferred mesophilic DNA polymerases include Pol I family of DNA polymerases (and their respective Klenow fragments) any of which may be isolated from organisms such as E. coli, H. influenzae, D. radiodurans, H. pylori, C. aurantiacus, R. prowazekii, T. pallidum, Synechocystis sp., B. subtilis, L. lactis, S. pneumoniae, M. tuberculosis, M. leprae, M. smegmatis, Bacteriophage L5, phi-C31, T7, T3, T5, SP01, SP02, mitochondrial from S. cerevisiae MIP-1, and eukaryotic C. elegans, and D. melanogaster (Astatke, M. et al., 1998, J. Mol. Biol. 278, 147-165), and Family A, Family B, Family C and pol III type DNA polymerase isolated for any sources, and mutants, derivatives or variants thereof, and the like. Preferred thermostable DNA polymerases that may be used in the methods and compositions of the invention include Taq, Tne, Tma, Pfu, Tfl, Tth, Stoffel fragment, VENT™ and DEEPVENT™ DNA polymerases, and mutants, variants and derivatives thereof which have preferably been modified such that they are more temperature sensitive and possess reduced, substantially reduced, or no polymerase activity and, optionally, reduced, substantially reduce or no exonuclease activity (U.S. Pat. No. 5,436,149; U.S. Pat. No. 4,889,818; U.S. Pat. No. 4,965,188; U.S. Pat. No. 5,079,352; U.S. Pat. No. 5,614,365; U.S. Pat. No. 5,374,553; U.S. Pat. No. 5,270,179; U.S. Pat. No. 5,047,342; U.S. Pat. No. 5,512,462; WO 92/06188; WO 92/06200; WO 96/10640; Barnes, W. M., Gene 112:29-35 (1992); Lawyer, F. C., et al., PCR Meth. Appl. 2:275-287 (1993); Flaman, J.-M, et al., Nucl. Acids Res. 22(15):3259-3260 (1994)).

In reducing, substantially reducing or eliminating polymerase activity, any one or a number of mutations in the polymerase domain of the polypeptide of interest which provides the desired result can be used. The sequence of many polymerases, in particular, Pol I Family (Type A) polymerases are known and the polymerase domain of such polymerase has been determined (Table 1, below), as well as the polymerase domain of bacteriophage RB69 polymerase (Wang, J. et al., 1997, Cell 89, 1087-1099). For other polymerases, one can readily locate the region corresponding to the polymerase domain using available sequence alignment data (Wang, J. et al., 1997, Cell 89, 1087-1099; Hopfner, K. et al. 1999, Proc. Natl. Acad. Sci. 96, 3600-3605; Braithwaite, D. and Ito, J., 1993, Nucleic Acids Res. 21, 787-802).

TABLE 1
E. coli Pol I family (Type A) Polymerase Polymerase
domain (Approximate Amino Acid Range)

| | |
|---|---|
| Pol I (E. coli) | 520-928 |
| Taq Pol | 424-831 |
| Tne Pol | 486-893 |
| Tth Pol | 426-834 |
| Tma Pol | 486-893 |
| Bst Pol | 472-879 |
| Bca Pol | 472-879 |
| T7 Pol | 200-704 |
| T5 Pol | 335-855 |

In addition, temperature sensitive (ts) mutants can be used in accordance with the invention. Ts mutants can be identified by assays well known in the art, for example, by determining the presence or absence of polymerase activity at elevated temperatures. The polymerase from E. coli ts mutant was identified and its sequence revealed a G544D mutation. By using sequence alignment, the amino acid from other Pol I family polymerases can be identified (Table 2) and used to make ts mutants at a position corresponding to this position. Polymerases with any other amino acid(s) that renders the polymerase temperature sensitive is contemplated in the present invention.

TABLE 2
Temperature sensitive mutations Polymerase Mutation

| | |
|---|---|
| E. coli Pol I | G544D |
| Tne Pol | G510 |
| Taq Pol | G448 |
| Tma Pol | G510 |
| Tth Pol | G450 |
| Bca Pol | G495 |
| Bst Pol | G494 |
| T7 Pol | G231 |
| T5 Pol | G359 |

Preferably, the polypeptide of the invention comprises a Pol I type DNA polymerase such as Klenow fragment (see Joyce et al., J. Bio. Chem. (1982)257:1958-1964; Polesky et al., J. Biol. Chem. (1990) 265:14579-14591). The Klenow fragment can be altered by introducing mutations into the enzyme to reduce its polymerase and 3′ to 5′ exonuclease activities. For example, D355A reduces 3′ to 5′ activity by 10,000 fold (Derbyshire et al., 1991, EMBO J. 10, 17-24). Specific residues have been identified in the polymerase domain of DNA polymerase I of E. coli which can affect polymerase activity, such as Arg754, Lys758, Phe762, Tyr766, His 734, Gln849, His881, Glu883, Asp705, Asp882, Arg 668, and Glu710 to name a few, although deletion and insertion mutation may also be used. Polymerase activity can be reduced by altering one or more residues in the polymerase domain, although deletion and insertion mutation may also be used. In addition, other residues in or outside of the polymerase domain, or deletion of a subdomain, may affect polymerase activity and would be useful in the present invention. D882A mutation in Klenow fragment reduces the polymerase activity by 1000-fold while increasing DNA affinity by 15-fold (Polesky et al., 1990, J. Biol. Chem. 265, 14579-14591). Additionally, mutants of Klenow fragment derivatives can also be made temperature sensitive. Mutations corresponding to these sites in other polymerases can be made for the purpose of reducing polymerase activity, increasing DNA affinity, reducing exonuclease activity, and/or rendering the polymerase temperature sensitive.

Reverse transcriptases for use in deriving the polypeptides of the invention include any enzyme having reverse transcriptase activity. Such enzymes include, but are not limited to, retroviral reverse transcriptase, retrotransposon reverse transcriptase, hepatitis B reverse transcriptase, cauliflower mosaic virus reverse transcriptase, bacterial reverse transcriptase, Tth DNA polymerase, Taq DNA polymerase (Saiki, R. K., et al., Science 239:487-491 (1988); U.S. Pat. Nos. 4,889,818 and 4,965,188), Tne DNA polymerase (WO 96/10640), Tma DNA polymerase (U.S. Pat. No. 5,374,553) and mutants, variants or derivatives thereof (see, e.g., WO 97/09451 and WO 98/47912). Preferred enzymes for use in the invention include those that have reduced, substantially reduced or eliminated RNase H activity. By an enzyme “substantially reduced in RNase H activity” is meant that the enzyme has less than about 20%, more preferably less than about 15%, 10% or 5%, and most preferably less than about 2%, of the RNase H activity of the corresponding wildtype or RNase H⁺ enzyme such as wildtype Moloney Murine Leukemia Virus (M-MLV), Avian Myeloblastosis Virus (AMV) or Rous Sarcoma Virus (RSV) reverse transcriptases. The RNase H activity of any enzyme may be determined by a variety of assays, such as those described, for example, in U.S. Pat. No. 5,244,797, in Kotewicz, M. L., et al., Nucl. Acids Res. 16:265 (1988) and in Gerard, G. F., et al., FOCUS 14(5):91 (1992), the disclosures of all of which are fully incorporated herein by reference. Particularly preferred polypeptides for use in the invention include, but are not limited to, M-MLV H⁻ reverse transcriptase, RSV H⁻ reverse transcriptase, AMV H⁻ reverse transcriptase, RAV (rous-associated virus) H⁻ reverse transcriptase, MAV (myeloblastosis-associated virus) H⁻ reverse transcriptase and HIV H reverse transcriptase (See U.S. Pat. No. 5,244,797 and WO 98/47912). It will be understood by one of ordinary skill, however, that any enzyme capable of producing a DNA molecule from a ribonucleic acid molecule (i.e., having reverse transcriptase activity) that is substantially reduced in RNase H activity may be equivalently used in the compositions, methods and kits of the invention. Preferred enzymes for use in the invention include those that are reduced or substantially reduced in polymerase activity. Such reduction of polymerase activity is preferably accomplished by any one or a number of mutations or modifications in the polymerase domain of the reverse transcriptase of interest using standard techniques. See, for example, WO 98/47912; and Shih-Fong et al. Nucleic Acid Res. (1995) 23:803-810.

Nucleases used to derive the polypeptides and the compositions of the invention include any protein or enzyme that has nuclease activity, but preferably includes single-strand specific exonucleases. Such exonucleases of the invention include, but are not limited to, any exonuclease (3′ to 5′ and 5′ to 3′ exonuclease) from any number of DNA polymerases such as Family A type DNA polymerases, Family B type DNA polymerases, Family C type DNA polymerases, pol III type DNA polymerases (e.g., episolin subunit), and pol I type DNA polymerases. Other exonucleases used in the invention include exo I, exo II, exo IV, exo V, exo VII, exo 31, espsilon subunit at DNA polymerase III, T4 exo IV, exonuclease from Bacillus, T5 exonuclease, lambda exonuclease, T7 exonuclease, RECJ exonuclease, exo II from yeast, exo V from yeast, phosphodiesterase, mammalian exo VII, exo IV from yeast, and exonuclease from Neurospora crassa. Examples of single-stranded and double-stranded exonucleases may be found for example in DNA replication (second edition) (A. Kornberg and T. A. Baker, DNA Replication, 2d ed., W.H. Freeman and Co, New York, 1992). Such nucleases/endonucleases of the invention include, but are not limited to, any endonucleases that cleave single stranded and/or double stranded nucleic acids such as RecBCD endonuclease, endonuclease I, endonuclease II and endonuclease VI from E. coli, T7 endonuclease, T4 endonuclease IV, micrococcal nuclease from Staphylococcus, Neurospora endonuclease, S1-nuclease from Aspergillus oryzae, PI-nuclease from Penicillium citrinum, Mung nuclease I, DNase 1, DNase II, AP endonucleases, Endo R, restriction endonucleases like EcoK (type I enzyme) and EcoRI (type II enzyme), repair endonucleases like T4 UV endo (endoV) and ribonucleases like RNase H. Examples of single-stranded and double stranded nucleases/endonucleases may be found for example in DNA Replication (second edition) (A. Kornberg and T. A. Baker, DNA Replication, 2 ed., W.H. Freeman and Co., New York, 1992). Nucleases for use in the invention also include RNase's and DNase's. See for example Nucleases, 2^{nd} ed, Ed. S. M. Lin, R. S. Lloyd, and R. J. Roberts, Cold Spring Harbor Laboratory Press, 1993. Examples of RNase's which may be used in the invention include RNase A, RNase H, RNase CL3, RNase PhyM, RNase T1, RNase T2 and RNase III. Examples of DNase's which may be used in the invention include DNase I and DNase II.

In reducing, substantially reducing or eliminating nuclease activity, any one or a number of mutations in the nuclease activity domain of the polypeptide of interest which provides the desired result can be used. The sequence of many nucleases or nuclease domains are known and the exonuclease domain has been determined (Table 3 below). For other nucleases, one can readily locate the region corresponding to the nuclease domain using available sequence or alignment data.

TABLE 3
Nucleases and Nuclease Domain

| | 3′ to 5′exo-domain | 5′ to 3′exo-domain |
|---|---|---|
| Polymerase | approx. amino acid range | approx. amino acid range |
| Pol I (E. coli) | 320-520 | 1-330 |
| Taq Pol | 290-425* | 1-300 |
| Tne Pol | 290-485 | 1-300 |
| Tth Pol | 290-425* | 1-300 |
| Tma Pol | 290-490 | 1-300 |
| Bst Pol | 290-475* | 1-300 |
| Bca Pol | 290-470* | 1-300 |
| T7 Pol | 1-200 | — |
| T5 Pol | 100-340 | — |

*Polymerases that have a putative 3′ to 5′ exo-domain but do not have measurable 3′ to 5′ exonuclease activity (deduced from sequence and structural comparisons). Each sequence represented with an asterisk is missing essential catalytic residues to have a detectable 3′ to 5′ exonuclease activity.

Additionally, one of ordinary skill in the art may make random mutations within the nuclease or enzyme of interest to inactivate the activity of the enzyme or protein (e.g., nuclease activity, polymerase activity or other activity of interest) using techniques well known in the art.

Polypeptides of the present invention are preferably used in the present compositions and methods at a final concentration in a synthesis, sequencing or amplification reaction sufficient to prevent or inhibit such synthesis, sequencing or amplification in the presence of a polymerase or reverse transcriptase enzyme. The ratio of inhibitory polypeptide of the invention to polymerase or reverse transcriptase may vary depending on the polymerase or reverse transcriptase and polypeptide used. The molar ratio of inhibitory peptide to polymerase/reverse transcriptase enzyme for a synthesis, sequencing or amplification reaction may range from about 0.001-100:1; 0.01-1000:1; 0.1-10,000:1; 1-100,000:1; 1-500,000:1; or 1-1,000,000:1. Of course, other suitable ratios of such inhibitory polypeptide to polymerase/reverse transcriptase suitable for use in the invention will be apparent to one or ordinary skill in the art or determined with no more than routine experimentation.

Methods of Nucleic Acid Synthesis

The polypeptides and compositions of the invention may be used in methods for the synthesis of nucleic acids. In particular, it has been discovered that the present polypeptides and compositions reduce nonspecific nucleic acid synthesis, particularly in amplification reactions such as the polymerase chain reaction (PCR). The present polypeptides and compositions may therefore be used in any method requiring the synthesis of nucleic acid molecules, such as DNA (including cDNA) and RNA molecules. Methods in which the polypeptides or compositions of the invention may advantageously be used include, but are not limited to, nucleic acid synthesis methods, nucleic acid amplification methods, including “hot-start” synthesis or amplification where the reaction is set up at a temperature below which the inhibitory polypeptide is inactivated or denatured and then the reaction is initiated by elevating the temperature to inactivate or denature the inhibitory polypeptide, thus allowing nucleic acid synthesis or amplification to take place.

Nucleic acid synthesis methods according to this aspect of the invention may comprise one or more steps. For example, the invention provides a method for synthesizing one or more nucleic acid molecules comprising (a) mixing one or more nucleic acid templates with one or more primers and the above-described polypeptides of the present invention and one or more enzymes having polymerase or reverse transcriptase activity to form a mixture; (b) incubating the mixture under conditions sufficient to inhibit nucleic acid synthesis; and (c) incubating the mixture under conditions sufficient to make one or more first nucleic acid molecules complementary to all or a portion of the templates. According to this aspect of the invention, the nucleic acid templates may be DNA molecules such as a cDNA molecule or library, or RNA molecules such as a mRNA molecule. Conditions sufficient to allow synthesis such as pH, temperature, ionic strength, and incubation times may be optimized according to the skill of people in the art.

Furthermore, the enzymes having polymerase activity for use in the invention may be obtained commercially, for example from Life Technologies, Inc. (Rockville, Md.), Perkin-Elmer (Branchburg, N.J.), New England BioLabs (Beverly, Mass.) or Boehringer Mannheim Biochemicals (Indianapolis, Ind.). Enzymes having reverse transcriptase activity for use in the invention may be obtained commercially, for example from Life Technologies, Inc. (Rockville, Md.), Pharmacia (Piscataway, N.J.), Sigma (Saint Louis, Mo.) or Boehringer Mannheim Biochemicals (Indianapolis, Ind.). Alternatively, polymerases or reverse transcriptases having polymerase activity may be isolated from their natural viral or bacterial sources according to standard procedures for isolating and purifying natural proteins that are well-known to one of ordinary skill in the art (see, e.g., Houts, G. E., et al., J. Virol. 29:517 (1979)). In addition, such polymerases/reverse transcriptases may be prepared by recombinant DNA techniques that are familiar to one of ordinary skill in the art (see, e.g., Kotewicz, M. L., et al., Nucl. Acids Res. 16:265 (1988); Soltis, D. A., and Skalka, A. M., Proc. Natl. Acad. Sci. USA 85:3372-3376 (1988)). Examples of enzymes having polymerase activity and reverse transcriptase activity may include any of those described in the present application which do not contain a mutation/modification to eliminate polymerase or reverse transcriptase activity.

In accordance with the invention, the input or template nucleic acid molecules or libraries may be prepared from populations of nucleic acid molecules obtained from natural sources, such as a variety of cells, tissues, organs or organisms. Cells that may be used as sources of nucleic acid molecules may be prokaryotic (bacterial cells, including those of species of the genera Escherichia, Bacillus, Serratia, Salmonella, Staphylococcus, Streptococcus, Clostridium, Chlamydia, Neisseria, Treponema, Mycoplasma, Borrelia, Legionella, Pseudomonas, Mycobacterium, Helicobacter, Erwinia, Agrobacterium, Rhizobium, and Streptomyces) or eukaryotic (including fungi (especially yeast's), plants, protozoans and other parasites, and animals including insects (particularly Drosophila spp. cells), nematodes (particularly Caenorhabditis elegans cells), and mammals (particularly human cells)).

Once the starting cells, tissues, organs or other samples are obtained, nucleic acid molecules (such as DNA, RNA (e.g., mRNA or poly A+ RNA) molecules) may be isolated, or cDNA molecules or libraries prepared therefrom, by methods that are well-known in the art (See, e.g., Maniatis, T., et al., Cell 15:687-701 (1978); Okayama, H., and Berg, P., Mol. Cell. Biol. 2:161-170 (1982); Gubler, U., and Hoffman, B. J., Gene 25:263-269 (1983)).

In the practice of a preferred aspect of the invention, a first nucleic acid molecule may be synthesized by mixing a nucleic acid template obtained as described above, which is preferably a DNA molecule or an RNA molecule such as an mRNA molecule or a polyA+ RNA molecule, with one or more of the above-described inhibitory polypeptides or compositions of the invention to form a mixture. Synthesis of a first nucleic acid molecule complementary to all or a portion of the nucleic acid template is preferably accomplished after raising the temperature of the reaction and denaturing or inactivating the inhibitory polypeptide of the present invention thereby freeing the nucleic acid synthesis substrate (e.g., double-stranded primer/template hybrid, and single-stranded primers and templates) and favoring the reverse transcription (in the case of an RNA template) and/or polymerization of the input or template nucleic acid molecule. Such synthesis is preferably accomplished in the presence of nucleotides (e.g., deoxyribonucleoside triphosphates (dNTPs), dideoxyribonucleoside triphosphates (ddNTPs) or derivatives thereof).

Of course, other techniques of nucleic acid synthesis in which the inhibitory polypeptides, compositions and methods of the invention may be advantageously used will be readily apparent to one of ordinary skill in the art.

Amplification and Sequencing Methods

In other aspects of the invention, the inhibitory polypeptides and compositions of the invention may be used in methods for amplifying or sequencing nucleic acid molecules. Nucleic acid amplification methods according to this aspect of the invention may additionally comprise use of one or more polypeptides having reverse transcriptase activity, in methods generally known in the art as one-step (e.g., one-step RT-PCR) or two-step (e.g., two-step RT-PCR) reverse transcriptase-amplification reactions. For amplification of long nucleic acid molecules (i.e., greater than about 3-5 Kb in length), a combination of DNA polymerases may be used, as described in WO 98/06736 and WO 95/16028.

Amplification methods according to this aspect of the invention may comprise one or more steps. For example, the invention provides a method for amplifying a nucleic acid molecule comprising (a) mixing a nucleic acid template with one or more of the inhibitory polypeptides or compositions of the invention to form a mixture; and (b) incubating the mixture under conditions sufficient to allow the enzyme with polymerase activity to amplify a nucleic acid molecule complementary to all or a portion of the template. In a preferred aspect, the conditions favoring synthesis inactivates or denatures the inhibitory polypeptide of the invention. The invention also provides nucleic acid molecules amplified by such methods.

General methods for amplification and analysis of nucleic acid molecules or fragments are well-known to one of ordinary skill in the art (see, e.g., U.S. Pat. Nos. 4,683,195; 4,683,202; and 4,800,159; Innis, M. A., et al., eds., PCR Protocols: A Guide to Methods and Applications, San Diego, Calif.: Academic Press, Inc. (1990); Griffin, H. G., and Griffin, A. M., eds., PCR Technology: Current Innovations, Boca Raton, Fla.: CRC Press (1994)). For example, amplification methods which may be used in accordance with the present invention include PCR (U.S. Pat. Nos. 4,683,195 and 4,683,202), Strand Displacement Amplification (SDA; U.S. Pat. No. 5,455,166; EP 0 684 315), and Nucleic Acid Sequence-Based Amplification (NASBA; U.S. Pat. No. 5,409,818; EP 0 329 822).

Typically, these amplification methods comprise: (a) contacting the nucleic acid sample with one or more inhibitory polypeptides or compositions of the present invention, one or more polypeptides having nucleic acid polymerase activity in the presence of one or more primer sequences, and (b) amplifying the nucleic acid sample to generate a collection of amplified nucleic acid fragments, preferably by PCR or equivalent automated amplification technique, and (c) optionally separating the amplified nucleic acid fragments by size, preferably by gel electrophoresis, and analyzing the gels for the presence of nucleic acid fragments, for example by staining the gel with a nucleic acid-binding dye such as ethidium bromide.

Following amplification or synthesis by the methods of the present invention, the amplified or synthesized nucleic acid fragments may be isolated for further use or characterization. This step is usually accomplished by separation of the amplified or synthesized nucleic acid fragments by size by any physical or biochemical means including gel electrophoresis, capillary electrophoresis, chromatography (including sizing, affinity and immunochromatography), density gradient centrifugation and immunoadsorption. Separation of nucleic acid fragments by gel electrophoresis is particularly preferred, as it provides a rapid and highly reproducible means of sensitive separation of a multitude of nucleic acid fragments, and permits direct, simultaneous comparison of the fragments in several samples of nucleic acids. One can extend this approach, in another preferred embodiment, to isolate and characterize these fragments or any nucleic acid fragment amplified or synthesized by the methods of the invention. Thus, the invention is also directed to isolated nucleic acid molecules produced by the amplification or synthesis methods of the invention.

In this embodiment, one or more of the amplified or synthesized nucleic acid fragments are removed from the gel which was used for identification (see above), according to standard techniques such as electroelution or physical excision. The isolated unique nucleic acid fragments may then be inserted into standard nucleotide vectors, including expression vectors, suitable for transfection or transformation of a variety of prokaryotic (bacterial) or eukaryotic (yeast, plant or animal including human and other mammalian) cells. Alternatively, nucleic acid molecules produced by the methods of the invention may be further characterized, for example by sequencing (i.e., determining the nucleotide sequence of the nucleic acid fragments), by methods described below and others that are standard in the art (see, e.g., U.S. Pat. Nos. 4,962,022 and 5,498,523, which are directed to methods of DNA sequencing).

Nucleic acid sequencing methods according to the invention may comprise one or more steps. For example, the invention provides a method for sequencing a nucleic acid molecule comprising (a) mixing a nucleic acid molecule to be sequenced with one or more primers, one or more of the above-described inhibitory polypeptides or compositions of the invention, one or more nucleotides, one or more terminating agents (such as a dideoxynucleotide), and one or more enzymes with polymerase activity to form a mixture; (b) incubating the mixture under conditions sufficient to synthesize a population of molecules complementary to all or a portion of the molecule to be sequenced; and (c) separating the population to determine the nucleotide sequence of all or a portion of the molecule to be sequenced. Nucleic acid sequencing techniques which may employ the present inhibitory polypeptides or compositions include dideoxy sequencing methods such as those disclosed in U.S. Pat. Nos. 4,962,022 and 5,498,523.

Vectors and Host Cells

The present invention also relates to vectors which comprise a nucleic acid molecule encoding one or more of the inhibitory polypeptides of the present invention such as a Klenow derivative as described herein. Further, the invention relates to host cells which contain the gene or genes encoding the polypeptides of the invention and preferably to host cells comprising recombinant vectors containing such gene or genes, and to methods for the production of the polypeptides of the invention using these vectors and host cells. Such host cells are preferably genetically engineered and used for production of recombinant polypeptides.

The vector used in the present invention may be, for example, a phage or a plasmid, and is preferably a plasmid. Preferred are vectors comprising cis-acting control regions to the nucleic acid encoding the polypeptide of interest. Appropriate trans-acting factors may be supplied by the host, supplied by a complementing vector or supplied by the vector itself upon introduction into the host.

In certain preferred embodiments in this regard, the vectors provide for specific expression of a polypeptide encoded by the nucleic acid molecules of the invention; such expression vectors may be inducible and/or cell type-specific. Particularly preferred among such vectors are those inducible by environmental factors that are easy to manipulate, such as temperature and nutrient additives.

Expression vectors useful in the present invention include chromosomal-, episomal-and virus-derived vectors, e.g., vectors derived from bacterial plasmids or bacteriophages, and vectors derived from combinations thereof, such as cosmids and phagemids.

The DNA insert should be operatively linked to an appropriate promoter, such as the phage lambda P_{L} promoter, the E. coli lac, trp and tac promoters. Other suitable promoters will be known to the skilled artisan. The gene fusion constructs will further contain sites for transcription initiation, termination and, in the transcribed region, a ribosome binding site for translation. The coding portion of the mature transcripts expressed by the constructs will preferably include a translation initiation codon at the beginning, and a termination codon (UAA, UGA or UAG) appropriately positioned at the end, of the polynucleotide to be translated.

The expression vectors will preferably include at least one selectable marker. Such markers include tetracycline or ampicillin resistance genes for culturing in E. Coli and other bacteria.

Among vectors preferred for use in the present invention include pQE70, pQE60 and pQE-9, available from Qiagen; pBS vectors, Phagescript vectors, Bluescript vectors, pNH8A, pNH16a, pNH18A, pNH46A, available from Stratagene; pcDNA3 available from Invitrogen; and pGEX, pTrxfus, pTrc99a, pET-5, pET-9, pKK223-3, pKK233-3, pDR540, pRIT5 available from Pharmacia. Other suitable vectors will be readily apparent to the skilled artisan.

Representative examples of appropriate host cells include, but are not limited to, bacterial cells such as E. coli, Streptomyces spp., Erwinia spp., Klebsiella spp. and Salmonella typhimurium. Preferred as a host cell is E. coli, and particularly preferred are E. coli strains DH10B and Stbl2, which are available commercially (Life Technologies, Inc; Rockville, Md.).

Peptide Production

As noted above, the methods of the present invention are suitable for production of any polypeptide of any length, via insertion of the above-described nucleic acid molecules or vectors into a host cell and expression of the nucleotide sequence encoding the polypeptide of interest by the host cell. Introduction of the nucleic acid molecules or vectors into a host cell to produce a transformed host cell can be effected by calcium phosphate transfection, DEAE-dextran mediated transfection, cationic lipid-mediated transfection, electroporation, transduction, transformation of chemically competent cells, infection or other methods. Such methods are described in many standard laboratory manuals, such as Davis et al., Basic Methods In Molecular Biology (1986). Once transformed host cells have been obtained, the cells may be cultivated under any physiologically compatible conditions of pH and temperature, in any suitable nutrient medium containing assimilable sources of carbon, nitrogen and essential minerals that support host cell growth. Recombinant polypeptide-producing cultivation conditions will vary according to the type of vector used to transform the host cells. For example, certain expression vectors comprise regulatory regions which require cell growth at certain temperatures, or addition of certain chemicals or inducing agents to the cell growth medium, to initiate the gene expression resulting in the production of the recombinant polypeptide. Thus, the term “recombinant polypeptide-producing conditions,” as used herein, is not meant to be limited to any one set of cultivation conditions. Appropriate culture media and conditions for the above-described host cells and vectors are well-known in the art. Following its production in the host cells, the polypeptide of interest may be isolated by several techniques. To liberate the polypeptide of interest from the host cells, the cells are lysed or ruptured. This lysis may be accomplished by contacting the cells with a hypotonic solution, by treatment with a cell wall-disrupting enzyme such as lysozyme, by sonication, by treatment with high pressure, or by a combination of the above methods. Other methods of bacterial cell disruption and lysis that are known to one of ordinary skill may also be used.

Following disruption, the polypeptide may be separated from the cellular debris by any technique suitable for separation of particles in complex mixtures. The polypeptide may then be purified by well known isolation techniques. Suitable techniques for purification include, but are not limited to, ammonium sulfate or ethanol precipitation, acid extraction, electrophoresis, immunoadsorption, anion or cation exchange chromatography, phosphocellulose chromatography, hydrophobic interaction chromatography, affinity chromatography, immunoaffinity chromatography, size exclusion chromatography, liquid chromatography (LC), high performance LC (BPLC), fast performance LC (FPLC), hydroxylapatite chromatography and lectin chromatography.

Kits

The present invention also provides kits for use in the synthesis, amplification, or sequencing of a nucleic acid molecule. Kits according to this aspect of the invention may comprise one or more containers, such as vials, tubes, ampules, bottles and the like, which may comprise one or more of the inhibitory polypeptides and/or compositions of the invention.

The kits of the invention may comprise one or more of the following components: (i) one or more polypeptides or compositions of the invention, (ii) one or more polymerases and/or reverse transcriptases, (iii) one or more suitable buffers, (iv) one or more nucleotides, and (v) one or more primers; (vi) one or more templates, and (vii) instructions for carrying out the methods of the invention.

Compositions

The present invention also relates to compositions prepared for carrying out the synthesis, amplification or sequencing methods of the invention and for carrying out the nuclease protection methods of the invention. Additionally, the invention relates to compositions made during or after carrying out such methods of the invention. In a preferred aspect, a composition of the invention comprise one or more of the inhibitory polypeptides of the invention. Such compositions may further comprise one or more components selected from the group consisting of: (i) one or more polymerases and/or reverse transcriptases, (ii) one or more suitable buffers, (iii) one or more nucleotides, (iv) one or more templates, (v) one or more primers, (vi) one or more templates/primer complexes, and (vii) one or more nucleic acid molecules made by the synthesis, amplification or sequencing methods of the invention.

The invention also relates to compositions comprising the polypeptides of the invention bound to or complexed with one or more nucleic acid molecules as well as the polypeptide(s)/nucleic acid molecule(s) complex found in such compositions or made during the methods of the invention.

It will be readily apparent to one of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

The following Materials and Methods were used in the Examples described below.

Cloning and protein preparation

I) Mutant A denotes a Klenow fragment derivative that carries one mutation (D882A) at the polymerase domain and two replacements at the 3′ to 5′ exo-nuclease domain (D355A and E357A): D882A reduces the polymerase activity by 600-fold (Polesky et al., 1990, supra). The combined mutation of D355A and E357A reduces 3′ to 5′ exonuclease activity to background level.

II) Mutant B denotes a Klenow fragment derivative that carries one mutation (D882N) at the polymerase domain and two replacements at the 3′ to 5′ exonuclease genotype as stated above. D882N reduces the polymerase activity by 10000-fold (Polesky et al., 1990, supra).

III) Mutant C denotes a Klenow fragment derivative that carries a double mutation (K758A and D882A) at the polymerase domain and the two replacements at the 3′ to 5′ exonuclease domain. Each of the individual substitution, D882A and K758A, reduce the polymerase activity by about 600-fold, respectively (Polesky et al., 1990; Astatke et al., 1995, J. Biol. Chem. 270, 1945-1954).

IV) Mutant D denotes a mutant Klenow fragment derivative that is derived from mutant A in a temperature sensitive background (polA12)-.

A thermostable reverse transcriptase enzyme that has been reported is a point mutant derivative of avain reverse transcriptase (RT), stable at 55° C. (e.g., Thermoscript™ II available from Life Technologies, Inc.; see also WO 98/47912). We propose here the use of a mutant derivative of Klenow fragment that exhibits the following phenotypes; inactive polymerase, binds DNA/DNA or DNA/RNA substrates and that is unstable above 37° C. in order not to compromise the RT catalyzed DNA synthesis. The purpose is to integrate such “PCR reagent” so as to reduce the level of non-specific DNA synthesis by a reverse transcriptase or polymerase during PCR or RT-PCR.

Engineering the D882A, D882N and K758A, Point Mutations.

The K758A, D882A and the D882N point mutations were engineered by site directed mutagenesis (SDM). A single stranded DNA was generated from the plasmid pTrcN2 having a Klenow fragment gene with two point mutations, D355A and E357A, inserted into the multiple cloning site. The oligonucleotides used for SDM to engineer the single point mutants were the following:

For D882A substitution, 5′ ATG ATC ATG CAG GTG CAT GCT GAA CTG GTA TTT G 3′ (SEQ ID NO:1) where a SphI site was created (bold italics).

For D882N substitution, 5′ ATG ATC ATG CAG GTG CAC AAC GAA CTG GTA TTT G 3′ (SEQ ID NO:2) where an ApaLI site was created (bold italics).

For K758A substitution, 5′ CAA CGC CGTAGC GCT GCA GCG ATC AAC TTT GG 3′ (SEQ ID NO:3) where a PstI site was created (bold italics).

The underlined codons denote positions that carry the mutations. The mutant containing the double replacement K758A and D882A (Mutant C) was created by doing an allelic exchange. The construct had a single MunI site down stream the codon for the amino acid at position 758 and a single HindIII site down stream the stop codon. The MunI-HindIII fragment from the construct K758A was replaced with the corresponding fragment from the D882A constructs in order to create a construct that was carrying both substitutions. The gene was under the control of the IPTG inducible Trc promoter.

Each of the construct was analyzed for the level of protein expression as follows: Overnight cultures were grown (2 ml) in Circle Grow (CG) (B1010, La Jolla, Calif.) containing ampicillin (100 mg/ml) at 30° C. To 40 ml of CG+Amp₁₀₀, 1 ml of the overnight culture was added and the culture was grown at 37° C. until it reached an O.D of about 1.0 (A₅₉₀). The culture was split into two 20 ml aliquots, and the first aliquot (uninduced) was kept at 37° C. To the other aliquot, IPTG was added to a final concentration of 2 mM and the culture was incubated at 37° C. After 3 hours the cultures were centrifuged at 4° C. in a table-top centrifuge at 3500 rpm for 20 minutes. The supernatant was poured off and the cell pellet was stored at −70° C. and the expressed protein was analyzed by SDS-PAGE. The cell pellet was suspended in 1 ml of buffer containing 10 mM Tris pH 8.0, 1 mM Na₂EDTA, 10 mM b-ME and was sonicated (Heat Systems). A 100 ml sample was kept for analysis of the total protein and the rest was centrifuged at 4° C. The supernatant was used for the analysis of the soluble proteins. Samples (amount equivalent to 0.1 A₅₉₀ units) were fractionated on a 4-20% gradient Tris-glycine gel, in the presence of b-ME in Tris-glycine SDS buffer.

In order to increase the expression of the protein, the mutated derivative of Klenow fragment were sub-cloned under the control of the 1 pL promoter. Following the digestion of the pTrcN2 construct with HindIII, the ends were filled by the wild-type Klenow fragment. Finally the construct was digested with NdeI and the fragment of approximately 1800 bp was sub-cloned into the vector pREI (Reddy et al., Nucleic Acid Res. (1989)17: 10473-10488) that had already been digested with NdeI and SmaI. The host for the construct used was DH10B (Life Technologies, Inc., Rockville, Md.) a host deficient in RNase I that carried the c1 repressor on a chloramphenicol (Cm) resistant plasmid. The level of protein expression was analyzed by SDS-PAGE as described above.

Overproduction and Purification of the Mutants of Klenow Fragment.

Cells were grown on a larger scale in shake flasks. For pTrcN2 constructs, 20 ml of CG+Amp₁₀₀ was inoculated using the glycerol seed. The culture was then grown overnight at 30° C. Ten ml of the overnight culture was added to a 500 mL of CG+Amp₁₀₀ mixture and was incubated at 37° C. Following cell growth (A₅₉₀ approximately 1.2) the cultures were induced with IPTG (2 mM final concentration) and were grown for three more hours. The cells were harvested by centrifugation and stored at −70° C.

For pRE1 constructs, 20 ml of CG+Amp₁₀₀+Cm₃₀ was inoculated with the glycerol seed. The culture was then grown at 30° C. overnight. A 7.5 ml of the overnight culture was added to 500 ml of CG+Amp₁₀₀+Cm₃₀ mixture and was incubated at 30° C. At cell density where the A₅₉₀ was about 1.2 the culture was induced by setting at 42° C. for 1 hour and then incubated at 37° C. for three hours. Finally, the cells were harvested by centrifugation and stored at −70° C.

All steps were carried out at 4° C. or on ice unless stated otherwise. The cells containing the recombinant plasmid (about 3 gms) were thawed and suspended in the sonication buffer (1:5 ratio of cells to buffer in 20 mM Tris pH7.5, 0.1 M KCl 1 mM Na₂EDTA, 1 mM DTT and 0.1 mM PMSF). The cell suspension was sonicated until greater than 80% of the total cell fraction was cracked open (determined by A₅₉₀ measurement). A solution of KCl (2M) was added to increase the concentration of KCl to 0.2M. This was followed by the dropwise addition of Polymin P (Sigma, St. Louis, Mo.) ( 1/9 volume of 5% v/v stock) with constant stirring and the suspension was stirred for an additional 20 minutes. The sample was then centrifuged at 10,000 rpm, 20 min, and the supernatant was fractionated by ammonium sulfate precipitation. The fraction precipitated by 40-55% ammonium sulfate was resuspended in 20 ml buffer containing 20 mM KPO₄pH 7.0, 0.1 M KCL, 1.5 M (NH₄)₂SO₄, 1 mM Na₂EDTA and 1 mM DTT (this is also the buffer used in the wash and gradient on the Butyl 650S column). The protein sample was loaded and chromatographed on a Butyl 650S column (Toxoltaas, Montgomeryville, Pa.) and was eluted by a linear gradient (20 mM KPO₄pH 7.0, 20% glycerol, 0.1 M KCl, 1 mM NA₂EDTA and 1 mM DTT). Fractions were analyzed by SDS-PAGE and those containing the mutant Klenow fragment were pooled.

The protein solution was dialyzed overnight against a buffer [20 mM KPO₄pH6.8, 0.1 M KCl, 1 mM DTT and 0.1 mM PMSF] and was then chromatographed on a hydroxyapatite column (AIC, Natick, Mass.), eluted using a linear gradient of phosphate from 20 mM to 250 mM. The fractions containing the mutant Klenow fragments were pooled and loaded on a cation exchange column (Fractogel EMD Sulfate (EM Separations, Wakefield R.I.)). The column was equilibrated and washed with a buffer [20 mM KPO₄ pH 6.5, 0.1 M KCl, 1 mM DTT and 0.1 mM PMSF], and was eluted using a linear gradient of KCl from 0.1 M to 0.75 M. The fractions containing the mutant Klenow fragment were pooled and dialyzed against buffer [50 mM KPO₄ pH7.0, 0.1 M KCl, 1 mM DTT and 50% glycerol].

### Example 1

The DNA polymerase activity of ThermoScript™ II RNase deficient mutant reverse transcriptase (RT) (available from Life Technologies, Inc., see also WO 98/47921) was determined at ambient temperature and 50° C. in the presence and absence of a Klenow fragment carrying mutations D355A, D357A, K758A, and D882A. The DNA substrate for the polymerase assay was a 34/60 mer primer/template. The 5′-terminus of the primer strand was labeled with ³²P using T4-polynucleotide kinase.

A polymerization reaction was initiated by the addition of RT/Klenow fragment solution (at different ratio) to a solution of the DNA substrate in the presence of DNTP and MgCl₂. The reaction concentration of the DNA was 0.5 nM to 2 nM, each of the four dNTP was 1 mM and [MgCl₂ and KCl] were 7.5 mM. For each reaction condition the concentration of RT was maintained at 190 nM whereas the concentration of the Klenow fragment ranged from 10 micromolar to 0. Four different ratio of mixes of KF-RT were tested for effective inhibition of DNA polymerase activity. The reactions were stopped at 1 and 6 minutes for each measurement.

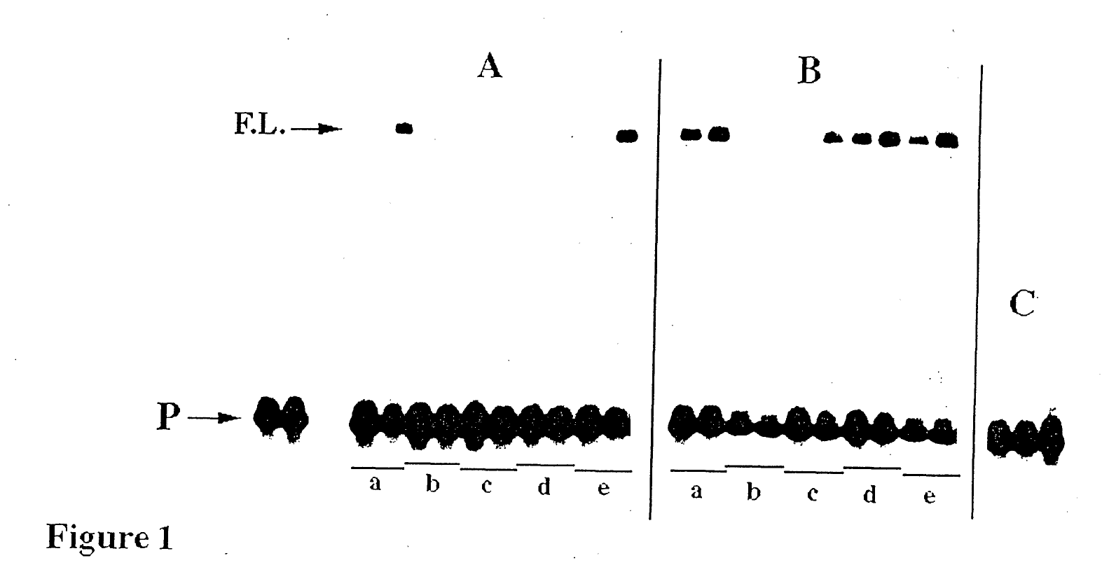

RT activity was inhibited at ambient temperature in the presence of a 5-fold (or more) excess of the Klenow fragment over RT under our reaction condition. However, at 50° C. RT competed for the DNA substrate detected by the significant DNA synthesis, even in the presence of a 50-fold excess KF (FIG. 1).

The Klenow fragment mutant derivative carrying the position replacements was assayed for polymerase activity so as to verify that the above mutations rendered the Klenow inactive with respect to its polymerase activity under the experimental conditions. There was an insignificant amount of polymerase activity even after 20 minutes incubation, at ambient temperature.

### Example 2

The activity of Taq, Tne, and KOD thermophylic DNA polymerases was determined at ambient temperature, 55° C. and 72° C. using the same DNA substrate described in example 1, in the presence and absence of the mutant Klenow fragment. For this assay, only a single Klenow fragment/active DNA polymerase ratio was assayed. The Klenow fragment was in excess so as to inhibit the polymerase activity at ambient temperature.

A polymerization reaction was initiated by the addition of one of the three DNA polymerases (in the presence or absence of the Klenow fragment) to a solution of the DNA substrate in the presence of DNTP and MgCl₂. The concentration of the DNA substrate was 0.5 nM, −2 nM each of the four dNTP was 200 um and [Mg²⁺] was 2 mM. The polymerization reactions were stopped at 1 and 4 minutes for measurements at ambient and 55° C., and only at 1 minute for determination at 72° C.

The polymerase activity of each of the thermophilic enzymes was significantly retarded at ambient temperature by the Klenow fragment. At 72° C., Klenow was not an effective inhibitor of the polymerase activity.

### Example 3

The 3′ to 5′ exo-nuclease activity of Tne DNA polymerase was measured using a single stranded 34-mer DNA substrate. The exonuclease directed DNA digestions were measured at ambient temperature, 37° C. and 72° C. in the presence and absence of the Klenow fragment (Mutant C). The 5′-terminus of the oligonucleotide substrate was labeled with ³²P using T4 polynucleotide kinase.

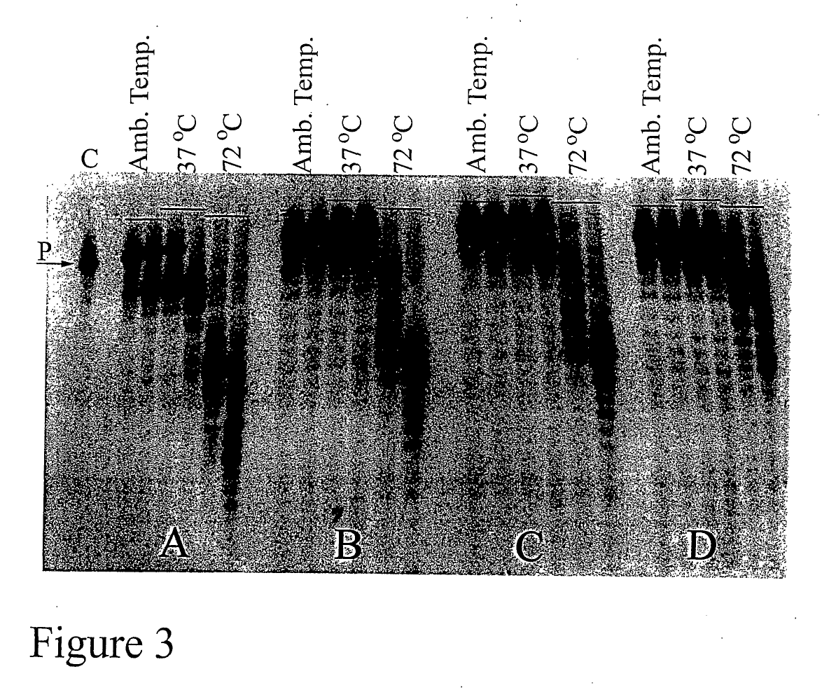

The exonuclease reaction was initiated by the addition of Tne DNA polymerase to a solution of the 34-mer substrate in the presence of Klenow fragment and MgCl₂. For the control reaction (see FIG. 3; panel A), Klenow fragment was not present. For each reaction, the reaction concentration of DNA substrate was 9 nM and the MgCl₇ was about 2 mM. The concentration of the Tne DNA polymerase was 60 nM, whereas, the concentration of the Klenow fragment varied from 0 to 20 μM.

The 3′ to 5′ exo-nuclease activity of Tne DNA polymerase was significantly inhibited at ambient temperature and 37° C. in the presence of the Klenow fragment. At 72° C., Klenow fragment was not a very effective inhibitor of the exo-nuclease activity.

Having now fully described the present invention in some detail by way of illustration and example for purposes of clarity of understanding, it will be obvious to one of ordinary skill in the ark that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

1-56. (canceled)

57. A method for amplifying double-stranded DNA molecule, comprising:
(a) providing a first and second primer, wherein said first primer is complementary to a sequence at or near the 3′-termini of the first strand of said DNA molecule and said second primer is complementary to a sequence at or near the 3′-termini of the second strand of said DNA molecule and art inhibitory polypeptide which has been mutated to substantially reduce or eliminate polymerase and/or exonuclease activity in said polypeptide, under conditions such that said polypeptide prevents or inhibits nucleic
(b) hybridizing said first primer to said first strand and said second primer to said second strand to form hybridized molecules;
(c) incubating said hybridized molecules under conditions sufficient to inactivate or denature said polypeptide and sufficient to allow synthesis of a third DNA molecule complementary to said first strand and a fourth DNA molecule complementary to said second strand;
(d) denaturing said first and third strand, and said second and fourth strands; and
(e) repeating steps (a) to (c) or (a) to (d) one or more times.

58. A method of preparing cDNA from mRNA, comprising
(a) forming a mixture by mixing one or more mRNA templates with an inhibitory polypeptide which has been mutated to substantially reduce or eliminate polymerase and/or exonuclease activity in said polypeptide; and
(b) incubating said mixture under conditions sufficient to synthesize a cDNA molecule complementary to all or a portion of said one or more templates, wherein said cDNA molecule is synthesized.

59. A method for amplifying a nucleic acid molecule comprising:
(a) forming a mixture by mixing one or more nucleic acid templates with an inhibitory polypeptide which has been mutated to substantially reduce or eliminate polymerase and/or exonuclease activity in said polypeptide under conditions sufficient to prevent or inhibit nucleic acid amplification; and
(b) incubating said mixture under conditions sufficient to inactivate or denature said polypeptide and sufficient to allow synthesis of a nucleic acid molecule complementary to all or a portion of said one or more templates,
wherein said nucleic acid molecule is amplified.


SEQUENCE LISTING (3 sequences)

SEQ ID NO: 1 - DNA, 34 bp, Artificial
  synthetic oligonucleotide
atgatcatgcaggtgcatgctgaactggtatttg

SEQ ID NO: 2 - DNA, 34 bp, Artificial
  synthetic oligonucleotide
atgatcatgcaggtgcacaacgaactggtatttg

SEQ ID NO: 3 - DNA, 32 bp, Artificial
  synthetic oligonucleotide
caacgccgtagcgctgcagcgatcaactttgg